(19) United States
(12) Patent Application Publication
(10) Pub. No.: US 2011/0306677 A1
(43) Pub. Date: Dec. 15, 2011

(54) Pressure Sensitive Adhesive Composition and Pressure Sensitive Adhesive Tape

(76) Inventor: Hiroshi Kataoka, Tokyo (JP)

(21) Appl. No.: 13/202,310

(22) PCT Filed: Feb. 15, 2010
(86) PCT No.: PCT/JP2010/052145

§ 371 Date: Aug. 18, 2011

(30) Foreign Application Priority Data
Feb. 20, 2009 (JP) 2009037372

(51) Int. Cl. A61K 31/045; A61K 47/32; B32B 7/12; C09J 153/00

(52) U.S. Cl. 514/729; 523/111; 514/772.1; 428/355 BL

(57) ABSTRACT

A pressure sensitive adhesive composition comprising, as a rubber component solid at ordinary temperature, from 10% by mass or more to less than 50% by mass of polyisoprene, from more than 50% by mass to 90% by mass or less of a styrene-isoprene-styrene block copolymer and 0 to 30% by mass of polyisobutylene, and further comprising, per 100 parts by mass of the solid rubber component, 1 to 30 parts by mass of polyisobutylene liquid at ordinary temperature, 50 to 120 parts by mass of a tackifier, and 1 to 40 parts by mass of a plasticizer liquid at ordinary temperature; and a pressure sensitive adhesive tape comprising a base material and the above-described pressure sensitive adhesive composition provided on the base material.

### TECHNICAL FIELD

The present invention relates to a pressure sensitive adhesive composition and a pressure sensitive adhesive tape with a layer of the pressure sensitive adhesive composition provided on a base material. More particularly, the present invention relates to a pressure sensitive adhesive composition that is excellent in suitability for coating, good in adhesive strength to the skin and adhesive strength to its own backing, little in stimulus to the skin and relieved in pain upon peeling, and a pressure sensitive adhesive tape having a layer of the pressure sensitive adhesive composition. The pressure sensitive adhesive tape according to the present invention is suitable for use in, for example, external patches for the skin, such as taping tapes.

### BACKGROUND ART

Uses of a pressure sensitive adhesive tape are developed as medical pressure sensitive adhesive products such as adhesive bandages, surgical pressure sensitive adhesive tapes and therapeutic pressure sensitive adhesive tapes making good use of its adhesive strength. For example, a taping tape is a pressure sensitive adhesive tape prepared for the purpose of fixing a soft vital tissue such as joint, muscle, ligament or tendon.

In sports, taping is conducted for the purpose of, for example, preventing an injury, taking an emergency measure or preventing recurrence. The prevention of the injury is conducted by making taping before an exercise such as a sport or training for preventing the injury in advance. The taping for the emergency measure is conducted for the purpose of taking an emergency measure against an injury caused by an exercise. The taping for the prevention of recurrence is conducted for preventing the injury from recurring.

Sports taping serves to prevent injury or the recurrence thereof by winding a taping tape on the joint, muscle, ligament, tendon or the like of a finger, hand, leg or the like to reinforce the site easy to be injured, or restrict the motion thereof. Sprain, injury of ligament, dislocation or the like is a lesion caused at the time the site to be injured has exceeded a normal range, and the restriction of the movable range by taping is effective for the prevention of such a lesion. The site injured in the past can also be reinforced and protected by taping, thereby relieving fear of injury.

The taping is spread in not only the field of sports, but also a taping field in everyday life for preventing or relieving various pains such as low back pain, acute low back pain, joint pains by injuries, and pains by hallux valgus. The taping is often conducted as an auxiliary means for rehabilitation for the purpose of supporting the injured site in a functional convalescence to relieve a pain.

The taping tape generally has a layer structure that a pressure sensitive adhesive layer is provided on one side of a base material and roughly divided into a non-expandable tape and an expandable tape. The taping tape is required to have great adhesive strength to the skin. On the other hand, the taping tape is also required to cause no pain upon peeling from the skin.

If the adhesive strength of the taping tape is too great, a rash or interruption in blood circulation is easy to occur, and a strong pain is easy to be caused upon peeling from the skin. If the adhesive strength of the taping tape is too small, the taping tape applied is easy to be peeled due to vigorous motion of the applied site by exercise or the like to fail to protect the applied site. In order to control the adhesive strength of the taping tape, it is considered that it is only necessary to select a pressure sensitive adhesive composition exhibiting moderate adhesive strength. However, the pain upon peeling from the skin does not always become strong in proportion to the adhesive strength of the taping tape. Therefore, it has been a difficult problem that both requirements of high adhesive strength to the skin and the relief of the pain upon peeling are satisfied.

In addition, the taping tape is required not to leave the pressure sensitive adhesive on the surface of the skin when peeled from the skin. The leaving (hereinafter referred to as “adhesive transfer”) of the pressure sensitive adhesive on the surface of the skin not only gives a user an unpleasant feeling, but also requires a post treatment for removing the pressure sensitive adhesive. The phenomenon of adhesive transfer does not appear in relation to the adhesive strength, and is greatly affected by adhesion property (anchorage property) to a base material and a pressure sensitive adhesive composition such as cohesion of the pressure sensitive adhesive.

In addition to these various properties, the taping tape is required to allow lap sticking. In the taping, a piece of the taping tape is wound on an ankle or the like, or plural pieces of the taping tape are stuck so as to overlap each other. Therefore, the taping tape is required to be not only excellent in tackiness to the skin, but also excellent in tackiness (hereinafter referred to as “tackiness to its own backing”) to the backing (a face of a base material, on which no pressure sensitive adhesive is provide, which is referred to as “its own backing”) of the tape itself.

As the base material of the taping tape, is generally used a fabric base material having flexibility and breathability. In order to enhance tackiness to the fabric base material (its own backing), it is effective to strengthen the adhesive strength of a pressure sensitive adhesive layer or make a pressure sensitive adhesive composition soft to lower cohesion of the resulting pressure sensitive adhesive. However, if the adhesive strength is too great, a pain becomes strong upon peeling of the tape from the skin on which the taping tape has been stuck. If the pressure sensitive adhesive composition is too soft, adhesive transfer on the surface of the skin is easy to occur upon peeling of the tape.

As described above, the taping tape is required to have conflicting various properties that both adhesive strength to the skin and adhesive strength to its own backing are great, and the pain upon peeling is weak and the adhesive transfer is also little.

Since motion pain often remains upon remedy using the taping tape, cooling or warming of the affected part is required together with fixation. For example, in an inflammatory phase from just after an injury to ordinarily 24 hours, in which the injury such as a bruise, blow or sprain is acute, it is necessary to cool the affected part together with rest. When the injury is chronic, and slight external force is applied repeatedly, remedy by warming is effective.

There is a method that an affected part is taped, and ice is then placed on the tape to cool the affected part. This cooling method is effective for just after injured or a heavy inflammatory phase. However, when cooling is required for a long period of time, the cooling by ice incur a great burden on the affected part. On the other hand, when a taping tape is applied on to an affected part, and a fabric poultice is then applied on to the taping tape, a poultice effect by percutaneous absorption cannot be achieved because a drug for poultice is hard to penetrate into the surface of the skin.

Since the taping tape is used for fixing an affected part, it is generally preset so as to have great adhesive strength. When such a taping tape is peeled, the peeling is heavy because the tape is strongly stuck on the skin, and so body hair is caught in the tape upon the peeling, or the skin is peeled off, followed by a violent pain. In order to relieve such a problem of the skin stimulus, it is known to stick an under-tape on the skin in advance and then conduct taping. However, this method is complicated in process.

As described above, the taping tape is required to have conflicting various properties that both adhesive strength to the skin and adhesive strength to its own backing are great, and the pain upon peeling is weak and the adhesive transfer is also little. In addition, it is necessary for the production of the taping tape to provide a pressure sensitive adhesive composition layer on a base material. Therefore, a pressure sensitive adhesive composition used is required to have excellent suitability for coating.

Japanese Utility Model Publication No. 6-50501 (Patent Literature 1) proposes a medical pressure sensitive adhesive tape obtained by fixing a breathable bag, in which an oxygen-reactive pyrogenic composition has been contained, to the surface of a heat-sealing resin layer of a laminate sheet of a material layer having oxygen gas barrier performance and the heat-sealing resin layer, laminating a laminate sheet having the same layer structure as the above-describe laminate sheet on the former laminate sheet with the heat-sealing resin layer inside, fusion-bonding peripheral portions of the two laminate sheets under heating and coating a back surface of the resultant laminate sheet with a pressure sensitive adhesive into which a drug has been incorporated. This medical pressure sensitive adhesive tape is used for hot therapy. However, it is difficult to take off clothes at the time the tape has been stuck because the thickness is great due to the multi-layer laminate sheet, and it is also difficult to lap-wind or spirally stick the tape for fixing the motion of a joint, so that the function inherent in the taping tape that the joint is fixed cannot be exhibited.

Japanese Patent Application Laid-Open No. 8-506127 (Patent Literature 2) through PCT route proposes a pressure-sensitive adhesive composition comprising solid rubber and liquid rubber at a weight ratio of 1:05 to 1:7 and applicable to the human skin. As examples of the solid rubber, SBR and A-B-A type block copolymer elastomers are mentioned. As examples of the liquid rubber, liquid isoprene rubber and liquid isoprene-styrene rubber are mentioned. The pressure-sensitive adhesive composition described in Patent Literature 2 is intended to develop tackiness by using the solid rubber and the liquid rubber in combination without incorporating a tackifying resin into a rubber component solid at ordinary temperature. Since the pressure-sensitive adhesive composition described in Patent Literature 2 contains a great amount of the liquid rubber, however, the composition has such defects that it gives a user a sticky feeling, cohesion is too low, it becomes too soft to easily cause cohesive failure and adhesive transfer to an adherend.

Japanese Patent Application Laid-Open No. 2002-69405 (Patent Literature 3) discloses a pressure sensitive adhesive composition comprising, per 100 parts by weight of a rubber component solid at ordinary temperature, 30 to 170 parts by weight of a rubber component liquid at ordinary temperature, and 50 to 400 parts by weight of a tackifier. As examples of the solid rubber component, A-B type or A-B-A type block copolymers are mentioned, and as examples of the liquid rubber component, liquid polyisoprene and liquid polybutadiene are mentioned. As examples of the tackifier, are shown tackifying resins of natural resin types such as terpene type and rosin type; and synthetic resin types such as aliphatic type, aromatic type, petroleum resin type, alkylphenol type, xylene type and coumarone-indene type. As examples of a base film, olefinic films are shown.

However, the pressure sensitive adhesive composition disclosed in Patent Literature 3 gives a user a feeling of hard pressure sensitive adhesion and a heavy peel feeling when the rubber solid at ordinary temperature is an A-B-A type block copolymer, and so skin stimuli such as a rash are easy to be incurred. This pressure sensitive adhesive composition is applied to uses of first-aid adhesive bandages and dressing materials, and not applied to taping tapes. In fact, a pressure sensitive adhesive tape obtained by providing a layer of this pressure sensitive adhesive composition on a base material is insufficient in cohesion and undergoes adhesive transfer, and a feeling upon use as a taping tape is unsatisfactory. In addition, to the pressure sensitive adhesive composition disclosed in Patent Literature 3 is applied a method that an organic solvent solution thereof is applied on to a separator and dried to form a pressure sensitive adhesive layer, and so its suitability for coating is unsatisfactory.

International Patent Publication No. WO2005/037946 Pamphlet (Patent Literature 4) discloses a pressure sensitive adhesive composition comprising polyisoprene, a styrene-isoprene-styrene copolymer and solid polyisobutylene at a weight ratio of 10-60:10-50:20-60 and further comprising, per 100 parts by weight in total of these 3 components, from 1 part by weight or more to less than 30 parts by weight of non-solid isobutylene and a tackifier. However, a pressure sensitive adhesive tape obtained by providing a layer of this pressure sensitive adhesive composition on a base material is still strong in pain upon peeling in addition to insufficient adhesive strength.

Japanese Patent Application Laid-Open No. 2006-55411 (Patent Literature 5) discloses a rubber type pressure sensitive adhesive composition obtained by incorporating 10 to 40% by mass of a functional group-modified liquid rubber component into a rubber type pressure sensitive adhesive composed of an elastomer component composed of natural rubber and/or diene type synthetic rubber, a tackifying resin, and a hydrophilic polymer component. However, the pressure sensitive adhesive composition disclosed in Patent Literature 5 and a pressure sensitive adhesive tape using this composition are applied to uses such as first-aid adhesive bandages and dressing materials, are not used as a taping tape and do not have the various properties required of the taping tape.

Japanese Patent Application Laid-Open No. 2006-206454 (Patent Literature 6) discloses a patch obtained by providing an ointment layer containing a rubber type pressure sensitive adhesive, a tackifier, a liquid additive and a styrene type thermoplastic elastomer on at least one side of a base material layer. A feature of this patch resides in that cold flow is inhibited, and a balance between cohesion and adhesion property is excellent. However, the pressure sensitive adhesive composition disclosed in Patent Literature 6 and a pressure sensitive adhesive tape using this composition are not used as a taping tape and do not have the various properties required of the taping tape. In addition, Patent Literature 6 shows that an organic solvent solution of the pressure sensitive adhesive composition was applied on to a release liner to form a pressure sensitive adhesive composition layer, and so the pressure sensitive adhesive composition is not excellent in suitability for coating.

### CITATION LIST

### Patent Literature

Patent Literature 1: Japanese Utility Model Publication No. 6-50501
Patent Literature 2: Japanese Patent Application Laid-Open No. 8-506127
Patent Literature 3: Japanese Patent Application Laid-Open No. 2002-69405
Patent Literature 4: International Patent Publication No. WO2005/037946
Patent Literature 5: Japanese Patent Application Laid-Open No. 2006-55411
Patent Literature 6: Japanese Patent Application Laid-Open No. 2006-206454

### SUMMARY OF INVENTION

### Technical Problem

A problem of the present invention is to provide a pressure sensitive adhesive composition that is excellent in suitability for coating, good in adhesive strength to the skin and adhesive strength to its own backing, little in stimulus to the skin and relieved in pain upon peeling, and a pressure sensitive adhesive tape having a layer of the pressure sensitive adhesive composition.

In particular, a problem of the present invention is to provide a pressure sensitive adhesive composition excellent in the above-described various properties and suitable for use in taping tapes, and a pressure sensitive adhesive tape having a layer of the pressure sensitive adhesive composition.

The present inventor has carried out an extensive investigation with a view toward solving the above problems. As a result, it has been found that the above problems can be solved by a pressure sensitive adhesive composition comprising, as a rubber component solid at ordinary temperature, polyisoprene, a styrene-isoprene-styrene block copolymer and polyisobutylene at a specific mass ratio, and further comprising polyisobutylene liquid at ordinary temperature, a tackifier, and a plasticizer liquid at ordinary temperature at specific proportions.

As the tackifier, a terpene type resin and an aliphatic hydrocarbon resin are preferably used in combination at a specific mass ratio. The liquid plasticizer preferably comprises liquid paraffin. The pressure sensitive adhesive composition according to the present invention may contain a hot point stimulant or cold point stimulant as a local stimulant. The pressure sensitive adhesive composition according to the present invention is excellent in suitability for coating and can be applied on to a base material or release liner by a calender coating method or hot-melt coating method without using a solution coating method to form a layer of the pressure sensitive adhesive composition. The present invention has been led to completion on the basis of these findings.

### Solution to Problem

According to the present invention, there is thus provided a pressure sensitive adhesive composition comprising, as a rubber component solid at ordinary temperature, from 10% by mass or more to less than 50% by mass of polyisoprene, from more than 50% by mass to 90% by mass or less of a styrene-isoprene-styrene block copolymer and 0 to 30% by mass of polyisobutylene, and further comprising, per 100 parts by mass of the solid rubber component, 1 to 30 parts by mass of polyisobutylene liquid at ordinary temperature, 50 to 120 parts by mass of a tackifier, and 1 to 40 parts by mass of a plasticizer liquid at ordinary temperature.

According to the present invention, there is also provided a pressure sensitive adhesive tape comprising a base material and a layer of the above-described pressure sensitive adhesive composition provided on the base material.

### Advantageous Effects of Invention

According to the present invention, there can be provided a pressure sensitive adhesive composition excellent in balance between adhesive strength and cohesion, good in adhesive strength to its own backing, relieved in pain upon peeling and excellent in suitability for coating. The pressure sensitive adhesive composition according to the present invention is excellent as a medical pressure sensitive adhesive tape and particularly suitable for use in taping tapes.

### DESCRIPTION OF EMBODIMENTS

The rubber components that are used in the present invention and solid at ordinary temperature are all rubber components which do not exhibit fluidity or semi-fluidity at ordinary temperature (20±15° C.) and can hold a solid form. The pressure sensitive adhesive composition according to the present invention comprises, as a rubber component solid at ordinary temperature, from 10% by mass or more to less than 50% by mass of polyisoprene, from more than 50% by mass to 90% by mass or less of a styrene-isoprene-styrene block copolymer and 0 to 30% by mass of polyisobutylene (the total of the 3 components is 100% by mass).

Polyisoprene used in the present invention is synthetic polyisoprene (cis-1,4-polyisoprene). Polyisoprene is a high-molecular weight polymer and rubber exhibiting a solid form at ordinary temperature. The Mooney viscosity (ML₁₊₄, 100° C.) of polyisoprene is generally 40 to 96, preferably 50 to 90. The number average molecular weight of polyisoprene is within a range of generally from 100,000 to 2,500,000, preferably from 500,000 to 2,000,000. The number average molecular weight is a value measured in terms of standard polystyrene by gel permeation chromatography (GPC).

Examples of commercially available polyisoprene include IR2200, product of JSR Corporation; NIPOL IR2000, NIPOL IR2200L and NIPOL IR2205 (trademark), products of ZEON CORPORATION; IR-307 and IR-310, products of KRATON POLYMERS JAPAN; and IR-10, product of Kuraray Co., Ltd. These products may be used either singly or in any combination thereof.

The styrene-isoprene-styrene block copolymer used in the present invention is generally a thermoplastic elastomer having a styrene content of 10 to 30% by mass and a melt flow rate (measured at a temperature of 200° C. under a load of 5 kg in accordance with JIS K 7210) of 1 to 20 g/10 min and solid at ordinary temperature. The number average molecular weight of the styrene-isoprene-styrene block copolymer as determined by the GPC method is within a range of from 45,000 to 400,000. Examples of commercially available styrene-isoprene-styrene block copolymers include KRATON D1107CP, D1107CU and D1161JP (trademark), products of KRATON POLYMERS JAPAN; SIS5000 and SIS5002, products of JSR Corporation; and QUINTAC 3421, 3460, 3520, 3530, 3620 and 3570C (trademark), products of ZEON CORPORATION. These products may be used either singly or in any combination thereof.

Polyisobutylene that is used in the present invention and solid at ordinary temperature is a high-molecular weight elastomer exhibiting elasticity. The viscosity-average molecular weight of the solid polyisobutylene as determined on the basis of the relationship between an intrinsic viscosity [η] and a molecular weight in diisobutylene is within a range of generally from 200,000 to 1,500,000, preferably from 300,000 to 1,300,000. Examples of commercially available solid polyisobutylene include OPPANOL B 100 (trademark), product of BASF Co.; and VISTANEX MML-80, VISTANEX MML-100 and VISTANEX MML-140 (trademark), products of Exxon Mobil Co. These products may be used either singly or in any combination thereof.

The pressure sensitive adhesive composition according to the present invention comprises, as the solid rubber component, from 10% by mass or more to less than 50% by mass of polyisoprene, from more than 50% by mass to 90% by mass or less of a styrene-isoprene-styrene block copolymer and 0 to 30% by mass of polyisobutylene and contains the styrene-isoprene-styrene block copolymer at a high proportion. Therefore, the pressure sensitive adhesive composition according to the present invention is suitable for coating by melting under heating. Examples of a coating method for the pressure sensitive adhesive composition according to the present invention include a hot-melt method and a calender method.

In order to relieve a pain upon peeling, the styrene-isoprene-styrene block copolymer having strong cohesion and high-molecular weight solid polyisobutylene are used in combination at the specific proportions, thereby improving the cohesion of the pressure sensitive adhesive composition and lessening the entering of the pressure sensitive adhesive composition into concave portions of the skin. When the proportion of the styrene-isoprene-styrene block copolymer is increased, the resulting pressure sensitive adhesive composition tends to enhance the cohesion and become hard. When the proportions of polyisoprene and solid polyisobutylene are increased, the resulting pressure sensitive adhesive composition tends to become soft.

On the other hand, polyisoprene rubber is contained in the pressure sensitive adhesive composition, whereby the adhesive strength to its own backing and adhesive strength to the skin of a pressure sensitive adhesive tape obtained by using this pressure sensitive adhesive composition can be improved. The adhesive strength of the pressure sensitive adhesive composition can be controlled within a desired range by suitably incorporating low-molecular weight liquid polyisobutylene and the tackifier. Therefore, the proportions of the rubber components solid at ordinary temperature are preferably determined from the viewpoints of balancing various properties such as pressure sensitive adhesion properties, suitability for processing and cohesion with one another at a high level.

The proportions of the rubber components solid at ordinary temperature are preferably (1) from 15% by mass or more to less than 50% by mass of polyisoprene, from more than 50% by mass to 85% by mass or less of the styrene-isoprene-styrene block copolymer and 0 to 25% by mass of polyisobutylene, more preferably (2) from 17% by mass or more to less than 50% by mass of polyisoprene, from more than 50% by mass to 83% by mass or less of the styrene-isoprene-styrene block copolymer and 0 to 23% by mass of polyisobutylene, particularly preferably (3) 18 to 49% by mass of polyisoprene, 51 to 82% by mass of the styrene-isoprene-styrene block copolymer and 0 to 20% by mass of polyisobutylene.

In the present invention, the rubber component solid at ordinary temperature may be composed of 2 components of polyisoprene and the styrene-isoprene-styrene block copolymer. When the rubber component solid at ordinary temperature is composed of the above-described 2 components, the proportions thereof are those that the proportion of polyisobutylene is subtracted from the total proportion of the above-described 3 components, and are (1) from 10% by mass or more to less than 50% by mass of polyisoprene and from more than 50% by mass to 90% by mass or less of the styrene-isoprene-styrene block copolymer, preferably (2) from 15% by mass or more to less than 50% by mass of polyisoprene and from more than 50% by mass to 85% by mass or less of the styrene-isoprene-styrene block copolymer, more preferably (3) from 17% by mass or more to less than 50% by mass of polyisoprene and from more than 50% by mass to 83% by mass or less of the styrene-isoprene-styrene block copolymer, particularly preferably (4) 18 to 49% by mass of polyisoprene and 51 to 82% by mass of the styrene-isoprene-styrene block copolymer (the total of the 2 components is 100% by mass).

When the rubber component solid at ordinary temperature is composed of the above-described 3 components, the proportion of polyisobutylene solid at ordinary temperature is preferably 1 to 30% by mass, more preferably 2 to 25% by mass, still more preferably 5 to 23% by mass, particularly preferably 7 to 20% by mass (the total of the 3 components is 100% by mass). In many cases, good results can be yielded when the proportion of polyisobutylene solid at ordinary temperature is 8 to 20% by mass.

In the proportion of polyisobutylene is too low, and the proportion of the styrene-isoprene-styrene block copolymer is too high, the resulting pressure sensitive adhesive composition becomes too hard, thereby adversely affecting a pressure sensitive adhesive tape obtained from such a pressure sensitive adhesive composition in pressure sensitive adhesion properties and a feeling upon use. If the proportion of polyisoprene is too low, the resulting pressure sensitive adhesive tape shows a tendency to lower the adhesive strength to its own backing and the adhesive strength to the skin.

If the proportion of polyisoprene is too high, and the proportion of the styrene-isoprene-styrene block copolymer is too low, the cohesion of the resulting pressure sensitive adhesive composition is lowered, and adhesive transfer is easy to occur. If the proportion of the styrene-isoprene-styrene block copolymer is too low, the resulting pressure sensitive adhesive composition shows a tendency to increase a pain upon peeling of a pressure sensitive adhesive tape obtained from such a composition in addition to an ill balance between adhesive strength and probe tack in the pressure sensitive adhesive composition.

Polyisobutylene solid at ordinary temperature is used, whereby the pressure sensitive adhesive composition can be made soft and a feeling of soft application and peeling can be obtained. If the proportion of solid polyisobutylene is too low, the function achieved by the use of the solid polyisobutylene cannot be attained. If the proportion of solid polyisobutylene is too high, the proportion(s) of polyisoprene and/or the styrene-isoprene-styrene block copolymer becomes too low, thereby lowering the pressure sensitive adhesion properties and cohesion of the resulting pressure sensitive adhesive composition.

The pressure sensitive adhesive composition according to the present invention can be synthetically improved in various properties such as pressure sensitive adhesion properties, releasability from the skin, adhesive strength to its own backing, adhesion property to its own backing and pain upon peeling by containing polyisobutylene liquid at ordinary temperature, a tackifier and a plasticizer liquid at ordinary temperature at specific proportions in the above-described solid rubber component. Since the pressure sensitive adhesive composition according to the present invention is excellent in the above-described various properties, it can be suitably applied to medical pressure sensitive adhesive tapes such as taping tapes.

Polyisobutylene liquid at ordinary temperature that is used in the present invention is generally a viscous liquid at ordinary temperature (20±15° C.) and widely includes low-molecular weight polyisobutylene which exhibits an amorphous form fluidic or semi-fluidic at ordinary temperature and cannot hold a fixed solid form. The viscosity-average molecular weight of the liquid polyisobutylene as determined on the basis of the relationship between an intrinsic viscosity [η] and a molecular weight in diisobutylene according to the Flory method or FCC method (FDA) is within a range of generally from 30,000 to 100,000, preferably from 35,000 to 90,000, particularly preferably from 40,000 to 70,000. As such low-molecular weight liquid polyisobutylene, a commercially available product may be used.

Examples of commercially available liquid polyisobutylene include VISTANEX LM-MS and VISTANEX LM-MH (trademark), products of Exxon Mobil Co.; OPPANOL B10SFN, OPPANOL B12SFN and OPPANOL B13SFN (trademark), products of BASF Co.; TETRAX 4T, TETRAX 5T and TETRAX 6T (trademark), products of Nippon Petrochemicals Co., Ltd.; and HIMOL 4H, HIMOL 5H, HIMOL 5.5H and HIMOL 6H (trademark), products of Nippon Petrochemicals Co., Ltd. These products may be used either singly or in any combination thereof.

Polyisobutylene liquid at ordinary temperature is used at a proportion of 1 to 30 parts by mass, preferably 3 to 28 parts by mass, more preferably 5 to 25 parts by mass to 100 parts by mass of the rubber component solid at ordinary temperature. Liquid polyisobutylene and the tackifier are contained in the pressure sensitive adhesive composition according to the present invention, whereby adhesive strength to bakelite and probe tack can be balanced with each other at a high level, thereby developing good pressure sensitive adhesion properties to the skin. If the proportion of liquid polyisobutylene is too high, the cohesion of the resulting pressure sensitive adhesive composition is lowered though adhesive strength and tack can be increased.

As the tackifier used in the present invention, are preferably a terpene type resin and an aliphatic hydrocarbon resin. When these two tackifying resins are used in combination, adhesive strength to the skin can be improved even when the cohesion of the rubber component solid at ordinary temperature is enhanced. The mass ration of the terpene resin to the aliphatic hydrocarbon resin is preferably within a range of from 1:1 to 5:1. The softening point of the terpene resin and aliphatic hydrocarbon resin is preferably within a range of from 65 to 130° C.

Examples of the terpene resin include YS RESIN PX and YS RESIN PXN (trademark), products of Yasuhara Chemical Co., Ltd.; and PICOLITE, (Trademark), product of Rika Fine-Tech Inc. Examples of the aliphatic hydrocarbon resin include QUINTONE A100, QUINTONE B170, QUINTONE K100, QUINTONE M100, QUINTONE R100 and QUINTONE C200S (trademark), products of ZEON CORPORATION. These products may be used either singly or in any combination thereof.

Other tackifiers may be used in combination as needed so far as no detrimental influence is thereby imposed on the pressure sensitive adhesion properties of the pressure sensitive adhesive composition according to the present invention. Examples of other tackifiers include rosin type resins [ESTER GUM (trademark), product of Arakawa Chemical Industries, Ltd.; HARIESTER (trademark), product of Harima Chemicals, Inc.; HARITAC (trademark), product of Harima Chemicals, Inc.], phenol type resins, xylene type resins, alkylphenol type resins, and coumarone-indene type resins. These tackifiers may be used either singly or in any combination thereof. The proportion of other tackifiers used is preferably 50 parts by mass or less, more preferably 30 parts by mass or less, particularly preferably 20 parts by mass or less per 100 parts by mass of the solid rubber component.

Alicyclic petroleum resins and alicyclic hydrogenated petroleum reins may also be used as tackifiers. In this case, however, the adhesive strength of the resulting pressure sensitive adhesive composition becomes too high to give a great pain upon peeling from the skin though the sticking ability thereof becomes good. Therefore, when such a tackifier is used, it is preferred that the proportion of the tackifier used is made extremely low.

The proportion of tackifier is 50 to 120 parts by mass, preferably 55 to 110 parts by mass, more preferably 60 to 100 parts by mass, particularly preferably 65 to 90 parts by mass per 100 parts by mass of the rubber component solid at ordinary temperature. If the proportion of the tackifier is too low, the resulting pressure sensitive adhesive composition shows a tendency to lower the adhesive strength and tack. The adhesive strength and tack are improved as the proportion of the tackifier increases. If the proportion becomes extremely high, however, the pressure sensitive adhesion properties at a low temperature of the resulting pressure sensitive adhesive composition is lowered and there is a tendency to give a pain upon peeling from the skin.

The plasticizer (liquid plasticizer) liquid at ordinary temperature exhibits an excellent plasticizing effect. As the liquid plasticizer, liquid paraffin is particularly preferred in that it is excellent in compatibility with the solid rubber component. The liquid plasticizer plays a part of controlling viscoelasticity of the pressure sensitive adhesive, enhancing adhesive strength to the skin and relieving the pain upon peeling by controlling its proportion to the solid rubber component. The pressure sensitive adhesive composition according to the present invention does preferably substantially not contain a plasticizer (solid plasticizer) solid at ordinary temperature.

When the pressure sensitive adhesive tape is provided as a percutaneous absorption preparation, other liquid plasticizers may be used for the purpose of improving percutaneous absorption property and storage stability and enhancing solubility of a drug in the pressure sensitive adhesive composition.

Examples of other plasticizers than liquid paraffin include fatty acid esters composed of a higher fatty acid having 12 to 16 carbon atoms and a lower monohydric alcohol having 1 to 4 carbon atoms, such as isopropyl myristate, ethyl laurate and isopropyl palmitate; fatty acids having 8 to 10 carbon atoms; glycols such as ethylene glycol, diethylene glycol, triethylene glycol, polyethylene glycol, propylene glycol and polypropylene glycol; oils and fats such as olive oil, castor oil, squalene and lanoline; organic solvents such as ethyl acetate, ethyl alcohol, dimethyldecyl sulfoxide, decylmethyl sulfoxide, dimethyl sulfoxide, dimethylformamide, dimethylacetamide, dimethyllaurylamide, dodecylpyrrolidone, isosorbitol, oleyl alcohol and lauric acid; liquid surfactants; and ethoxylated stearyl alcohol, glycerol esters, isotridecyl myristate, N-methylpyrrolidone, ethyl oleate, oleic acid, diisopropyl adipate, octyl palmitate, 1,3-propanediol and glycerol. A compound liquid at ordinary temperature is used among these compounds.

The other liquid plasticizers may be used either singly or in any combination thereof. Among the other liquid plasticizers, glycerol esters are preferred, middle chain fatty acid triglycerides that are esters of a fatty acid preferably having 8 to 10 carbon atoms and glycerol are more preferred. Examples of the middle chain fatty acid triglycerides include glyceryl tri(caprylate/caprate).

When the pressure sensitive adhesive composition and pressure sensitive adhesive tape are used as medical pressure sensitive adhesive tapes such as taping tapes, liquid paraffin and other liquid plasticizer(s) are preferably used in combination as the liquid plasticizers. When liquid paraffin and other liquid plasticizer(s) are used in combination, the proportion of liquid paraffin in the liquid plasticizers is preferably 40% by mass or more, more preferably 45% by mass or more, and the upper limit thereof is preferably 90% by mass. When liquid paraffin and a middle chain fatty acid triglyceride are used in combination, a mass ratio between both components is particularly preferably within a range of from 1:1 to 5:1.

The liquid plasticizer is used at a proportion of 1 to 40 parts by mass, preferably 3 to 38 parts by mass, more preferably 5 to 35 parts by mass, particularly preferably 8 to 33 parts by mass per 100 parts by mass of the solid rubber component. If the proportion of the liquid plasticizer is too low, it is difficult to exhibit its function. If the proportion is too high, the resulting pressure sensitive adhesive composition becomes too flexible to low the cohesion thereof, so that it is difficult to achieve satisfactory pressure sensitive adhesion properties. If the proportion of the liquid plasticizer is too high, the fluidity of the resulting pressure sensitive adhesive composition at a temperature near to the body temperature becomes high, the base material is easy to be separated due to the fluidity thereof, and the pressure sensitive adhesive composition enters into fine concave portions of the skin to give a pain upon peeling.

As described above, according to the present invention, there can be obtained a pressure sensitive adhesive composition synthetically improved in suitability for coating, cohesiveness, adhesive strength, adhesive strength to its own backing, adhesion property to its own backing, pain upon peeling and the like by containing the liquid polyisobutylene, tackifier and liquid plasticizer in the rubber component composed of the specific combination of the solid rubbers, and excellent in a feeling upon use, and a pressure sensitive adhesive tape having a pressure sensitive adhesive layer composed of the pressure sensitive adhesive composition.

The terpene type resin and aliphatic hydrocarbon resin are used in combination as the tackifier, whereby a pressure sensitive adhesive composition having good pressure sensitive adhesion properties to the skin can be obtained even when the cohesion of the solid rubber component is improved. In addition, the liquid plasticizer is contained, whereby the solid rubber component may be composed of 2 components of polyisoprene and the styrene-isoprene-styrene block copolymer. The pressure sensitive adhesive tape using the pressure sensitive adhesive composition according to the present invention may be given functions such as improved suitability for coating, improved adhesive strength of the pressure sensitive adhesive composition to the skin, relieving of pain upon peeling and good sticking ability to the skin greatly varied among individuals by making up the solid rubber component with the 2 components of polyisoprene and the styrene-isoprene-styrene block copolymer.

The pressure sensitive adhesive composition according to the present invention may contain a drug. No particular limitation is imposed on the drug so far as it is a drug having percutaneous absorption property. Examples of the drug include skin stimulants, analgesic-antiinflammatory drugs, antimycotic drugs, central nervous acting drugs, diuretics, hypotensive drugs, coronary vasodilators, antitussive-expectorants, antihistaminics, antiarrhythmic drugs, cardiotomics, contraceptives, adrenocorticotropic hormone antagonists or local anesthetics, and vitamins.

Examples of the skin stimulants and analgesic-antiinflammatory drugs include salicylic acid, methyl salicylate, glycol salicylate, acetylsalicylic acid, L-menthol, camphor (D-form, L-form, DL-form), peppermint oil, thymol, benzyl nicotinate, pepper extract, capsaicin, nonylic acid vanillylamide, felbinac, butyl flufenamate, piroxicam, indometacin, ketoprofen, pranoprofen, feprazone, flurbiprofen, loxoprofen, amfenac sodium, oxaprozine, emorfazone, thiaprofen, fenbufen, fentiazac, dichlorofenac sodium, diflunisal, ibuprofen piconol, bendazac, sprofen, buprenorphine hydrochloride, pentazocine and butorphanol tartrate.

The above-mentioned drugs may be used either singly or in any combination thereof. When the pressure sensitive adhesive tape is applied to the use of a taping tape, nonylic acid vanillylamide and L-menthol are preferably used as a warm spot stimulant and a cold spot stimulant, respectively. When L-menthol is contained in the pressure sensitive adhesive composition the composition gives a user a fresh feeling, and a function of relieving unpleasant odor by sweats or the like is imparted to the composition. Although menthol may be contained in the base material, menthol is preferably contained in the pressure sensitive adhesive composition for causing the skin to feel fresh.

The proportion of nonylic acid vanillylamide is preferably 0.0001 to 0.1% by mass, more preferably 0.005 to 0.05% by mass based on the total amount of the composition. The proportion of L-menthol is preferably 1 to 20% by mass, more preferably 3 to 18% by mass based on the total amount of the composition. Since the pressure sensitive adhesive composition according to the present invention is excellent in the balance between adhesive strength and cohesion, the pressure sensitive adhesive tape can develop a function as a taping tape even when L-menthol is contained in a relatively great amount, and it is hard to cause a practical problem.

Since the pressure sensitive adhesive composition according to the present invention is excellent in the balance among the various properties, it is unnecessary to incorporate a filler for the purpose of lowering adhesive strength or cohesion. Examples of the filler unnecessary to be incorporated include calcium carbonate, magnesium carbonate, hydrous silica, silica, zinc oxide, synthetic aluminum silicate and aluminum hydroxide. When the pressure sensitive adhesive composition is intended to prevent coloring or oxidation, various kinds of fillers may be incorporated in a proportion that physicochemical properties are not adversely affected, preferably in a proportion of 3% by mass or less based on the total amount of the pressure sensitive adhesive composition.

Various kinds of additive components such as dissolving agents, solubilizers, age resistors, antioxidants, preservatives, ultraviolet absorbents, colorants, perfume bases, surfactants and pH adjustors may be incorporated into the pressure sensitive adhesive composition according to the present invention as necessary for the end application intended.

The base material of the pressure sensitive adhesive tape according to the present invention is suitably selected according to uses thereof. Examples of the base material include films, sheets or metal foils of polyolefins such as polyethylene, polypropylene and ethylene-vinyl acetate copolymers; polybutadiene; polyvinyl chloride; thermoplastic polyesters such as polyethylene terephthalate, polybutylene terephthalate and polyethylene naphthalate; nylon; polyurethane; cotton; rayon (cellulose derivatives); or aluminum foil. An expandable or non-expandable base material such as a porous body or foam composed of any of these polymers, paper, woven fabric, knitted fabric, or nonwoven fabric may also be used. A laminate of these materials may also be used as the base material.

The pressure sensitive adhesive composition and pressure sensitive adhesive tape according to the present invention may be used in, for example, percutaneous absorption patches, first-aid adhesive bandages, or surgical dressing materials for the purpose of protecting a wound after a surgical operation; tapes surgical drapes for the purpose of preventing an incised part from being contaminated with bacteria; tapes for reinforcement and fixation of a sutured incision; and taping tapes used upon sports by selecting the base material.

In the use in the taping tape in particular, since the pressure sensitive adhesive tape is often wound on a hand, leg or the like, a flexible base material having properties such as flexibility, expandability, hand cutability and pressure sensitive adhesion to its own backing is preferably used. For example, when a woven fabric is used as the base material, a woven fabric obtained by arranging a composite yarn composed of cellulosic fiber and elastic fiber in a part or the whole of warp, or woven fabric obtained by alternately arranging a yarn hardly twisted in an S-Z direction of a cellulosic fiber yarn in warp is preferred. The cellulosic fiber means a raw material undergoing degradation of a part of a constituent molecular chain by a treatment with a natural cellulose-decomposing enzyme to lower its average polymerization degree, such as cotton, viscose rayon or polynogic fiber. The composite yarn composed of cellulosic fiber and elastic fiber means a twisted union yarn, covered yarn or core spun yarn of cellulosic fiber and fiber (for example, polyether type polyurethane fiber, polyester type polyurethane fiber or synthetic rubber fiber) having elasticity. Among these, core spun yarn of polyether type polyurethane fiber and cellulosic fiber is preferred. however, the composite yarn is not limited thereto. The mixed proportions of the cellulosic fiber and elastic fiber are preferably 85 to 98% by mass, more preferably 90 to 95% by mass for the cellulosic fiber.

A yarn used in a weft direction may be any fiber of the so-called synthetic fibers such as polyester fibers, polyamide fibers and acrylic fibers, and cellulosic fibers such as cotton and rayon.

A release liner may be arranged on the pressure sensitive adhesive composition layer of the pressure sensitive adhesive tape according to the present invention as needed. The release liner is selected from films, sheets or foils of polyolefins such as polyethylene and polypropylene; thermoplastic polyesters such as polyethylene terephthalate and polyethylene naphthalate; nylon; and aluminum, or paper, and a laminate of these materials may also be used. The surface of the release liner may be surface-treated with silicone or the line for making separation of the pressure sensitive adhesive composition layer easy.

The production processes of the pressure sensitive adhesive composition and pressure sensitive adhesive tape according to the present invention may be suitably selected from the conventionally performed processes. As a specific example thereof, a drug and other additive components are added and mixed into a pressure sensitive adhesive composition dissolved or kneaded, and the resultant mixture is directly spread on a base material, or once spread on a release liner such as paper or a film, which has been subjected to a releasing treatment, and the mixture spread is then applied and transferred to a base material, whereby a pressure sensitive adhesive tape can be produced. The pressure sensitive adhesive composition according to the present invention is particularly suitable for use in coating by a calender method. When solid polyisobutylene is not incorporated as a solid rubber component, coating by a hot melt method may also be selected.

The thickness of the pressure sensitive adhesive composition layer in the pressure sensitive adhesive tape according to the present invention is generally 30 to 400 μm, preferably 50 to 200 μm. If the thickness of the pressure sensitive adhesive composition layer is too great, the resulting pressure sensitive adhesive tape tends to cause adhesive transfer on the skin after stuck or worsen a release rate of the drug contained in the pressure sensitive adhesive composition. If the thickness of the pressure sensitive adhesive composition layer is too small, the resulting pressure sensitive adhesive tape tends to become poor in adhesion property to the skin to form the cause of falling off.

### EXAMPLES

The present invention will hereinafter be described more specifically by Examples and Comparative Examples. However, the present invention is not limited to these examples. Evaluation methods of properties of pressure sensitive adhesive tape samples obtained in respective Examples and Comparative Examples are as follows.

### (1) Adhesive Strength to Bakelite

A pressure sensitive adhesive tape sample having a width of 15 mm was stuck on a bakelite panel under an atmosphere of 23° C. in temperature in accordance with JIS Z 0237 prescribed in Japanese Industrial Standards, and they were bonded under pressure by reciprocating a rubber roll of 2 kg at a rate of 300 mm/min once on the tape. After left to stand for 20 minutes in this state, peel strength was measured under conditions of a peel angle of 180° and a peel rate of 300 mm/min. This peel strength is referred to as adhesion strength (unit: N/25 mm) to bakelite. The adhesive strength to bakelite is ranked as good where the peel strength at 180° to the bakelite panel falls within a range of from 3 to 10 N/25 mm

### (2) Probe Tack

A probe tack tester manufactured by NICHIBAN CO., LTD. was used to measure a probe tack (unit: N/5 mm in diameter) of a pressure sensitive adhesive tape sample in accordance with the probe tack testing method described for reference in JIS Z 0237. The probe tack is ranked as good where the value thereof falls with a range of from 2 to 6 N/5 mm in diameter.

### (3) Adhesive Strength to its Own Backing

A pressure sensitive adhesive tape sample 1 cut into a strip having a length of 100 mm and a width of 25 mm was stuck on a bakelite panel. A pressure sensitive adhesive tape sample 2 cut into a strip having a length of 100 mm and a width of 15 mm was lap-stuck on the base material (backing) of the pressure sensitive adhesive tape sample 1 with its end portion left. The tape samples were then bonded under pressure by reciprocating a rubber roll of 2 kg at a rate of 300 mm/min once on the base material of the pressure sensitive adhesive tape sample 2 under an atmosphere of 23° C. in temperature in accordance with JIS Z 0237. After left to stand for 20 minutes in this state, peel strength of the pressure sensitive adhesive tape sample 2 to the base material (backing) of the pressure sensitive adhesive tape 1 was measured under conditions of a peel angle of 180° and a peel rate of 300 mm/min. This peel strength is referred to as adhesion strength (unit: N/25 mm) to its own backing.

### (4) Adhesion Property to its Own Backing

A pressure sensitive adhesive tape sample having a width of 50 mm and a length of 250 mm was stuck on a movable part of each wrist in adult 5 men while winding and lap-sticking the tape sample. After about 6 hours, the applied state of the lap-stuck part just before peeling was evaluated according to the following standard to determine an average value, thereby ranking an evaluated result as A where a score obtained by rounding off the average value to the first decimal place was 4, B where the score was 3, C where the score was 2, or D where the score was 1.

Score 4: Not peeled at all;
Score 3: Partially peeled, but not such that a problem is caused;
Score 2: Peeled, and a problem is offered in holding of the lap-stuck state; and
Score 1: Completely peeled.

### (5) Pain Upon Peeling

A pressure sensitive adhesive tape sample having a width of 50 mm and a length of 250 mm was stuck on a movable part of each wrist in adult 5 men while winding and lap-sticking the tape sample. After about 6 hours, pain upon peeling when the tape sample was peeled form the skin was evaluated according to the following standard to determine an average value, thereby ranking an evaluated result as A where a score obtained by rounding off the average value to the first decimal place was 4, B where the score was 3, C where the score was 2, or D where the score was 1.

Score 4: Scarcely feel pain;
Score 3: Somewhat feel pain, but no problem is offered;
Score 2: Feel pain, and a problem is offered; and
Score 1: Feel strong pain.

### Example 1

Into 100 parts by mass of a solid rubber component composed of 30 parts by mass of polyisoprene [NIPOL IR2200 (trademark), product of ZEON CORPORATION], 51 parts by mass of a styrene-isoprene-styrene block copolymer [KRATON D1107CP (trademark), product of KRATON POLYMERS JAPAN] and 19 parts by mass of solid polyisobutylene [OPPANOL B100 (trademark), product of BASF], were added and melt-mixed 10 parts by mass of liquid polyisobutylene [HIMOL 4H (trademark), product of Nippon Petrochemicals Co., Ltd], 55 parts by mass of a terpene type resin [YS RESIN PX1150N (trademark), products of Yasuhara Chemical Co., Ltd.] and 20 parts by mass of an aliphatic hydrocarbon resin [QUINTONE B 170 (trademark), product of ZEON CORPORATION].

Into the composition obtained above, were added and mixed 25 parts by mass of liquid paraffin [HICALL M352 (trademark), product of KANEDA Co., Ltd.], 5 parts by mass of a middle chain fatty acid triglyceride [TRIESTER F810 (trademark), product of Nikko Chemicals Co., Ltd.] and 2 parts by mass of dibutylhydroxytoluene [antioxidant; IRGANOX 565 (trademark), product of Ciba Specialty Chemicals Co.] while heating and melting at 100° C. L-Menthol was then added in such a manner that the proportion thereof is 12% by mass. The thus-obtained pressure sensitive adhesive composition was spread on one side of a woven fabric (UK7032, product of Toyobo STC Co., Ltd.) having a weight of 150 g/m² so as to give a thickness of 70 μm, and the thus-treated woven fabric was wound on a roll with a width of 5 cm to produce a pressure sensitive adhesive tape. The results are shown in Table 1.

### Example 2

A pressure sensitive adhesive composition and a pressure sensitive adhesive tape were produced in the same manner as in Example 1 except that the proportions of solid polyisoprene and the styrene-isoprene-styrene block copolymer were changed from 30 parts by mass to 21 parts by mass and from 51 parts by mass to 60 parts by mass, respectively, as shown in Table 1. The results are shown in Table 1.

### Example 3

A pressure sensitive adhesive composition and a pressure sensitive adhesive tape were produced in the same manner as in Example 1 except that the styrene-isoprene-styrene block copolymer was changed from KRATON D1107CP to KRATON D1107CU (product of KRATON POLYMERS JAPAN), solid polyisobutylene was changed from OPPANOL B100 to VISTANEX MML-120 (trademark), product of Exxon Mobil Co.), the proportion of the terpene type resin was changed from 55 parts by mass to 50 parts by mass, the proportion of liquid paraffin was changed from 25 parts by mass to 5 parts by mass, no L-menthol was added, and nonylic acid vanillylamide was added at a proportion of 0.01% by mass as shown in Table 1. The results are shown in Table 1.

### Example 4

A pressure sensitive adhesive composition and a pressure sensitive adhesive tape were produced in the same manner as in Example 3 except that the proportion of liquid polyisobutylene was changed from 25 parts by mass to 20 parts by mass, the proportion of the terpene type resin was changed from 50 parts by mass to 55 parts by mass, and the proportion of liquid paraffin was changed from 5 parts by mass to 10 parts by mass as shown in Table 1. The results are shown in Table 1.

### Example 5

A pressure sensitive adhesive composition and a pressure sensitive adhesive tape were produced in the same manner as in Example 3 except that the proportion of liquid polyisobutylene was changed from 25 parts by mass to 10 parts by mass, the proportion of the terpene type resin was changed from 50 parts by mass to 55 parts by mass, and the proportion of liquid paraffin was changed from 5 parts by mass to 15 parts by mass as shown in Table 1. The results are shown in Table 1.

### Example 6

A pressure sensitive adhesive composition and a pressure sensitive adhesive tape were produced in the same manner as in Example 5 except that the proportion of the styrene-isoprene-styrene block copolymer was changed from 51 parts by mass to 60 parts by mass, and the proportion of solid polyisobutylene was changed from 19 parts by mass to 10 parts by mass as shown in Table 1. The results are shown in Table 1.

### Example 7

Into 100 parts by mass of a solid rubber component composed of 40 parts by mass of polyisoprene [NIPOL IR2200L (trademark), product of ZEON CORPORATION] and 60 parts by mass of a styrene-isoprene-styrene block copolymer [KRATON D1107CU (trademark), product of KRATON POLYMERS JAPAN], were added and melt-mixed 5 parts by mass of liquid polyisobutylene [HIMOL 4H (trademark), product of Nippon Petrochemicals Co., Ltd], 70 parts by mass of a terpene type resin [YS RESIN PX1150N (trademark), products of Yasuhara Chemical Co., Ltd.] and 20 parts by mass of an aliphatic hydrocarbon resin [QUINTONE B170 (trademark), product of ZEON CORPORATION].

Into the composition obtained above, were added and mixed 10 parts by mass of liquid paraffin, 10 parts by mass of a middle chain fatty acid triglyceride [TRIESTER F810 (trademark), product of Nikko Chemicals Co., Ltd.] and 2 parts by mass of dibutylhydroxytoluene while heating and melting at 100° C. Nonylic acid vanillylamide was then added in such a manner that the proportion thereof is 0.01% by weight based on the total weight of the composition to prepare a pressure sensitive adhesive composition. The thus-obtained pressure sensitive adhesive composition was spread on one side of a woven fabric (UK7032, product of Toyobo STC Co., Ltd.) having a weight of 150 g/m² so as to give a thickness of 80 μm, and the thus-treated woven fabric was wound on a roll with a width of 5 cm to produce a pressure sensitive adhesive tape. The results are shown in Table 1.

### Example 8

A pressure sensitive adhesive composition and a pressure sensitive adhesive tape were produced in the same manner as in Example 7 except that the proportion of the terpene type resin was changed from 70 parts by mass to 60 parts by mass as shown in Table 1. The results are shown in Table 1.

### Comparative Example 1

A pressure sensitive adhesive composition and a pressure sensitive adhesive tape were produced in the same manner as in Example 1 except that the proportion of polyisoprene was changed from 30 parts by mass to 40 parts by mass, the proportion of the styrene-isoprene-styrene block copolymer was changed from 51 parts by mass to 40 parts by mass, the proportion of solid polyisobutylene was changed from 19 parts by mass to 20 parts by mass, and the proportion of liquid paraffin was changed from 25 parts by mass to 15 parts by mass as shown in Table 1. The results are shown in Table 1.

### Comparative Example 2

A pressure sensitive adhesive composition and a pressure sensitive adhesive tape were produced in the same manner as in Comparative Example 1 except that the proportion of liquid paraffin was changed from 15 parts by mass to 45 parts by mass as shown in Table 1. The results are shown in Table 1.

TABLE 1

| | | | | | | | | | Comp. | |
|---|---|---|---|---|---|---|---|---|---|---|
| | Example | | | | | | | | Example | |
| | 1 | 2 | 3 | 4 | 5 | 6 | 7 | 8 | 1 | 2 |
| Polyisoprene (a) | 30 | 21 | 30 | 30 | 30 | 30 | — | — | 40 | 40 |
| Polyisoprene (b) | — | — | — | — | — | — | 40 | 40 | — | — |
| SIS (a) | 51 | 60 | — | — | — | — | — | — | 40 | 40 |
| SIS (b) | — | — | 51 | 51 | 51 | 60 | 60 | 60 | — | — |
| Polyisobutylene (a) | 19 | 19 | — | — | — | — | — | — | 20 | 20 |
| Polyisobutylene (b) | — | — | 19 | 19 | 19 | 10 | — | — | — | — |
| Liquid polyisobutylene | 10 | 10 | 25 | 20 | 10 | 10 | 5 | 5 | 10 | 10 |
| Tackifier | | | | | | | | | | |
| Terpene type resin | 55 | 55 | 50 | 55 | 55 | 55 | 70 | 60 | 55 | 55 |
| Aliphatic hydrocarbon resin | 20 | 20 | 20 | 20 | 20 | 20 | 20 | 20 | 20 | 20 |
| Liquid plasticizer | | | | | | | | | | |
| Liquid paraffin | 25 | 25 | 5 | 10 | 15 | 15 | 10 | 10 | 15 | 45 |
| Middle chain fatty acid triglyceride | 5 | 5 | 5 | 5 | 5 | 5 | 10 | 10 | 5 | 5 |
| Dibutylhydroxytoluene | 2 | 2 | 2 | 2 | 2 | 2 | 2 | 2 | 2 | 2 |
| L-Menthol (%) | 12 | 12 | — | — | — | — | — | — | 12 | 12 |
| Nonylic acid vanillylamide (%) | — | — | 0.01 | 0.01 | 0.01 | 0.01 | 0.01 | 0.01 | — | — |
| Thickness of pressure sensitive adhesive (μm) | 70 | 70 | 70 | 70 | 70 | 70 | 80 | 80 | 70 | 70 |
| Adhesive strength to bakelite (N/25 mm) | 3.14 | 3.35 | 9.62 | 9.33 | 8.03 | 6.32 | 8.09 | 8.42 | 4.49 | 2.77 |
| Adhesive strength to its own backing (N/25 mm) | 4.86 | 4.36 | 7.39 | 7.60 | 6.59 | 5.44 | 3.65 | 2.63 | 2.82 | 8.45 |
| Probe tack (N/5 mm in diameter) | 2.90 | 3.01 | 2.76 | 3.17 | 2.93 | 2.67 | 3.93 | 5.37 | 1.66 | 2.32 |
| Adhesion property to its own backing | A | A | B | A | B | B | A | B | A | B |
| Pain upon peeling | A | A | A | B | A | B | A | A | C | C |

(Note)
(1) Polyisoprene (a): polyisoprene rubber [NIPOL IR2200 (trademark), product of ZEON CORPORATION; Mooney viscosity (central value) = 83]
(2) Polyisoprene (b): polyisoprene rubber [NIPOL IR2200L (trademark), product of ZEON CORPORATION; Mooney viscosity (central value) = 70]
(3) SIS (a): styrene-isoprene-styrene block copolymer [KRATON D1107CP (trademark), products of KRATON POLYMERS JAPAN]
(4) SIS (b): styrene-isoprene-styrene block copolymer [KRATON D1107CU (trademark), products of KRATON POLYMERS JAPAN]
(5) Polyisobutylene (a): solid polyisobutylene [OPPANOL B100 (trademark), product of BASF]
(6) Polyisobutylene (b): solid polyisobutylene [VISTANEX MML-120 (trademark), products of Exxon Mobil Co.]
(7) Liquid polyisobutylene: HIMOL 4H (trademark), product of Nippon Petrochemicals Co., Ltd.; viscosity-average molecular weight = 40,000
(8) Terpene type resin: YS RESIN PX1150N (trademark), product of Yasuhara Chemical Co., Ltd.; softening point = 115 ± 5° C.
(9) Aliphatic hydrocarbon resin: QUINTONE B170 (trademark), products of ZEON CORPORATION; softening point = 70° C.
(10) Liquid paraffin: HICALL M352 (trademark), product of KANEDA Co., Ltd.
(11) Middle chain fatty acid triglyceride: glyceryl tri(caprylate/caprate); NIKKOL TRIESTER F810 (trademark), product of Nikko Chemicals Co., Ltd.
(12) Dibutylhydroxytoluene: antioxidant; IRGANOX 565 (trademark), Product of Ciba Specialty Chemicals Co.

### <Consideration>

A pressure sensitive adhesive tape stuck on the skin can be ranked as good in the pressure sensitive adhesion properties thereof where the peel strength at 180° to a bakelite panel falls within a range of from 3 to 9 N/25, and the probe tack falls within a range of from 2 to 6 N/5 mm in diameter. The pressure sensitive adhesive tapes of Examples 1 to 8 exhibit pressure sensitive adhesion properties within the preferable numerical ranges described above.

The adhesive strength to its own backing of a pressure sensitive adhesive tape is determined for examining sticking ability when the pressure sensitive adhesive tape is stuck on the surface of the base material (its own backing) of the pressure sensitive adhesive tape itself, and the tape is stuck over again after a short period of stuck time (about 20 minutes). The adhesion property to its own backing is determined for examining a peeled state after the pressure sensitive adhesive tape is actually wound on an arm and left to stand for a long period of stuck time (about 6 hours). The pain upon peeling is determined for examining a pain upon peeling when the pressure sensitive adhesive tape is stuck on the skin for 6 hours and then peeled from the skin.

The pressure sensitive adhesive tapes of Examples 1 to 8 are excellent in pressure sensitive adhesion properties, good in balance between adhesive strength to its own backing and adhesion property to its own backing and relieved in the pain upon peeling. On the other hand, the pressure sensitive adhesive tape of Comparative Example 1 is low in probe tack value, poor in balance between adhesive strength to bakelite and probe tack, low in initial (after about 20 minutes) adhesive strength to its own backing, shows a tendency to increase adhesion property to its own backing as the time goes on (after 6 hours) and gives a strong pain upon peeling. The pressure sensitive adhesive tape of Comparative Example 2 is low in adhesive strength to bakelite, shows a tendency to be extremely high in initial adhesive strength to its own backing but somewhat low in adhesion property to its own backing and gives a strong pain upon peeling.

As apparent from the above-described experimental data, a pressure sensitive adhesive tape excellent in pressure sensitive adhesion properties, good in balance between adhesive strength to bakelite and probe tack and greatly relieved in pain upon peeling can be obtained when the proportion of the respective components fall within the respective ranges of the present invention. The pressure sensitive adhesive compositions are also good in suitability for coating. The pressure sensitive adhesive compositions and pressure sensitive adhesive tapes according to the present invention are excellent in balance between adhesive strength and cohesion, good in a feeling upon use, relieved in pain upon peeling and suitable for medical pressure sensitive adhesive tapes. The pressure sensitive adhesive tapes according to the present invention allow lap-sticking in addition to the above-described properties and are thus suitable for use as taping tapes.

### Example 9

Into 100 parts by mass of a solid rubber component composed of 20 parts by mass of polyisoprene [NIPOL IR2200 (trademark), product of ZEON CORPORATION], 60 parts by mass of a styrene-isoprene-styrene block copolymer [KRATON D1161JP (trademark), product of KRATON POLYMERS JAPAN] and 20 parts by mass of solid polyisobutylene [OPPANOL B100 (trademark), product of BASF], were added and melt-mixed 10 parts by mass of liquid polyisobutylene [HIMOL 4H (trademark), product of Nippon Petrochemicals Co., Ltd], 40 parts by mass of a terpene type resin [YS RESIN PX1150N (trademark), products of Yasuhara Chemical Co., Ltd.] and 10 parts by mass of an aliphatic hydrocarbon resin [QUINTONE B 170 (trademark), product of ZEON CORPORATION].

Into the composition obtained above, were added and mixed 25 parts by mass of liquid paraffin [HICALL M352 (trademark), product of KANEDA Co., Ltd.], 5 parts by mass of a middle chain fatty acid triglyceride [TRIESTER F810 (trademark), product of Nikko Chemicals Co., Ltd.] and 5 parts by mass of dibutylhydroxytoluene [antioxidant; IRGANOX 565 (trademark), product of Ciba Specialty Chemicals Co.] while heating and melting at 100° C. L-Menthol was then added in such a manner that the proportion thereof is 8% by mass. The thus-obtained pressure sensitive adhesive composition was spread on one side of a woven fabric (UK7032, product of Toyobo STC Co., Ltd.) having a weight of 150 g/m² so as to give a thickness of 140 μm, and the thus-treated woven fabric was wound on a roll with a width of 5 cm to produce a pressure sensitive adhesive tape. The results are shown in Table 2.

### Example 10

A pressure sensitive adhesive tape was produced in the same manner as in Example 9 except that the proportions of the terpene type resin and the aliphatic hydrocarbon resin were changed from 40 parts by mass to 96 parts by mass and from 10 parts by mass to 24 parts by mass, respectively. The results are shown in Table 2.

### Example 11

A pressure sensitive adhesive tape was produced in the same manner as in Example 9 except that the proportions of the terpene type resin and the aliphatic hydrocarbon resin were changed from 40 parts by mass to 37.5 parts by mass and from 10 parts by mass to 37.5 parts by mass, respectively. The results are shown in Table 2.

### Example 12

A pressure sensitive adhesive tape was produced in the same manner as in Example 9 except that the proportions of the terpene type resin and the aliphatic hydrocarbon resin were changed from 40 parts by mass to 62.5 parts by mass and from 10 parts by mass to 12.5 parts by mass, respectively. The results are shown in Table 2.

TABLE 2

| | Example | | | |
|---|---|---|---|---|
| | 9 | 10 | 11 | 12 |
| Polyisoprene (a) | 20 | 20 | 20 | 20 |
| SIS (c) | 60 | 60 | 60 | 60 |
| Polyisobutylene (a) | 20 | 20 | 20 | 20 |
| Liquid polyisobutylene | 10 | 10 | 10 | 10 |
| Tackifier | | | | |
| Terpene type resin (T₁) | 40 | 96 | 37.5 | 62.5 |
| Aliphatic hydrocarbon resin (T₂) | 10 | 24 | 37.5 | 12.5 |
| Mass ratio (T₁/T₂) | 4/1 | 4/1 | 1/1 | 5/1 |
| Liquid plasticizer | | | | |
| Liquid paraffin | 25 | 25 | 25 | 25 |
| Middle chain fatty acid | 5 | 5 | 5 | 5 |
| triglyceride | | | | |
| Dibutylhydroxytoluene | 5 | 5 | 5 | 5 |
| L-Menthol (%) | 8 | 8 | 8 | 8 |
| Thickness of pressure sensitive | 140 | 140 | 140 | 140 |
| adhesive (μm) | | | | |
| Adhesive strength to bakelite | 3.58 | 12.89 | 5.42 | 6.14 |
| (N/25 mm) | | | | |
| Adhesive strength to its own | 3.13 | 9.76 | 4.53 | 5.17 |
| backing (N/25 mm) | | | | |
| Probe tack (N/5 mm in diameter) | 2.53 | 5.08 | 3.12 | 3.89 |
| Adhesion property to its own | B | A | B | A |
| backing | | | | |
| Pain upon peeling | A | B | B | B |

(Note)
(1) Polyisoprene (a): polyisoprene rubber [NIPOL IR2200 (trademark), product of ZEON CORPORATION; Mooney viscosity (central value) = 83]
(2) SIS (c): styrene-isoprene-styrene block copolymer [KRATON D1161JP (trademark), products of KRATON POLYMERS JAPAN]
(3) Polyisobutylene (a): solid polyisobutylene [OPPANOL B100 (trademark), product of BASF]
(4) Liquid polyisobutylene: HIMOL 4H (trademark), product of Nippon Petrochemicals Co., Ltd.; viscosity-average molecular weight = 40,000
(5) Terpene type resin: YS RESIN PX1150N (trademark), product of Yasuhara Chemical Co., Ltd.; softening point = 115 ± 5° C.
(6) Aliphatic hydrocarbon resin: QUINTONE B170 (trademark), products of ZEON CORPORATION; softening point = 70° C.
(7) Liquid paraffin: HICALL M352 (trademark), product of KANEDA Co., Ltd.]
(8) Middle chain fatty acid triglyceride: glyceryl tri(caprylate/caprate); NIKKOL TRIESTER F810 (trademark), product of Nikko Chemicals Co., Ltd.
(9) Dibutylhydroxytoluene: antioxidant; IRGANOX 565 (trademark), Product of Ciba Specialty Chemicals Co.

### <Consideration>

As apparent from the results shown in Table 2, it is understood that a pressure sensitive adhesive tape excellent in balance among pressure sensitive adhesion properties, adhesion property to its own backing and pain upon peeling and suitable for use as a medical pressure sensitive adhesive such as a taping tape can be obtained even when the terpene type resin (T₁) and the aliphatic hydrocarbon resin (T₂) are used as tackifiers in combination by changing the mass ratio (T₁/T₂) between them within a range of from 1:1 to 5:1.

### INDUSTRIAL APPLICABILITY

The pressure sensitive adhesive compositions according to the present invention can be used as pressure sensitive adhesives for pressure sensitive adhesive tapes. The pressure sensitive adhesive tapes according to the present invention can be used in, for example, a technical field of medical pressure sensitive adhesive tapes such as taping tapes.

1. A pressure sensitive adhesive composition comprising, as a rubber component solid at ordinary temperature, from 10% by mass or more to less than 50% by mass of polyisoprene, from more than 50% by mass to 90% by mass or less of a styrene-isoprene-styrene block copolymer and 0 to 30% by mass of polyisobutylene, and further comprising, per 100 parts by mass of the solid rubber component, 1 to 30 parts by mass of polyisobutylene liquid at ordinary temperature, 50 to 120 parts by mass of a tackifier, and 1 to 40 parts by mass of a plasticizer liquid at ordinary temperature.

2. The pressure sensitive adhesive composition according to claim 1, wherein the rubber component solid at ordinary temperature contains 18 to 49% by mass of polyisoprene, 51 to 82% by mass of the styrene-isoprene-styrene block copolymer and 0 to 20% by mass of polyisobutylene.

3. The pressure sensitive adhesive composition according to claim 1, wherein the polyisoprene solid at ordinary temperature is synthetic polyisoprene having a Mooney viscosity (ML₁₊₄, 100° C.) within a range of from 40 to 96 and a number average molecular weight within a range of from 100,000 to 2,500,000 in terms of standard polystyrene as measured by gel permeation chromatography.

4. The pressure sensitive adhesive composition according to claim 1, wherein the styrene-isoprene-styrene block copolymer solid at ordinary temperature has a melt flow rate within a range of from 1 to 20 g/10 min as measured at a temperature of 200° C. under a load of 5 kg in accordance with JIS K 7210 and a number average molecular weight within a range of from 45,000 to 400,000 in terms of standard polystyrene as measured by gel permeation chromatography.

5. The pressure sensitive adhesive composition according to claim 1, wherein the polyisobutylene solid at ordinary temperature has a viscosity-average molecular weight within a range of from 200,000 to 1,500,000.

6. The pressure sensitive adhesive composition according to claim 1, wherein the polyisobutylene liquid at ordinary temperature has a viscosity-average molecular weight within a range of from 30,000 to 100,000.

7. The pressure sensitive adhesive composition according to claim 1, wherein the tackifier comprises a terpene resin and an aliphatic hydrocarbon resin at a mass ratio ranging from 1:1 to 5:1.

8. The pressure sensitive adhesive composition according to claim 1, wherein the proportion of the tackifier is within a range of from 50 to 120 parts by weight per 100 parts by mass of the rubber component solid at ordinary temperature.

9. The pressure sensitive adhesive composition according to claim 1, wherein the plasticizer liquid at ordinary temperature comprises liquid paraffin.

10. The pressure sensitive adhesive composition according to claim 1, wherein the plasticizer liquid at ordinary temperature comprises liquid paraffin and a middle chain fatty acid triglyceride at a mass ratio ranging from 1:1 to 5:1.

11. The pressure sensitive adhesive composition according to claim 1, wherein the proportion of the plasticizer liquid at ordinary temperature is within a range of from 1 to 40 parts by weight per 100 parts by mass of the rubber component solid at ordinary temperature.

12. The pressure sensitive adhesive composition according to claim 1, which does substantially not comprise a plasticizer solid at ordinary temperature.

13. The pressure sensitive adhesive composition according to claim 1, which further comprises a warm spot stimulant or cold spot stimulant as a local stimulant.

14. The pressure sensitive adhesive composition according to claim 1, which further comprises, as a cold spot stimulant, L-menthol at a proportion ranging from 1 to 20% by mass based on the total mass of the pressure sensitive adhesive composition.

15. A pressure sensitive adhesive tape comprising a base material and a layer of the pressure sensitive adhesive composition according to claim 1 provided on the base material.
